SEQUENCE LISTING (4 sequences)

SEQ ID NO: 1 - DNA, 420 bp, Homo sapiens
attttttatacaccttatttgctcagacctgtaacttcagcctggagtgaacacagacacctagttttcctcaaactcctcttgggctttagagagaaggtgctggccctttgagccaagcaggttattggttagtagtacctctcagctctgaaggcttttcaatggtatatagcaacaagctgctctgtaagatagaggcagactgtcatcaggaatacctgtattcaccctgggaggcagctgaattccccaggatagattatgagatgctggaacattactgtaacgctggctttgcataaagatgggtcttgtcccattccttcaccagtaagctcttgtgggctgaacattgcaacaacagagctctcttcggttccaacgtgcgcgtaaatccacgagcagtagaaaaatgag

SEQ ID NO: 2 - DNA, 705 bp, Homo sapiens
taaacaggactatgtaatcaatcattttattattttcatcaacaaccaacacagcaacacaaattgacagccagtcctcaagggaaccttttttgaagtgaagagcaaggccctgaatgagctacctacatggaacatagtatttgcaatatgcaaatggagacctaaaggctcatgacgggaacagaaggtggctgtatacagaggaacagacacgaagtggagttatgggaagttcaatcaacaattggcaacaagctaggtcagtagtttacgaaagtggtcatctatacagtgcctgagtttgggttctgggcctggatcatgtaagaaaggcagcaaagtctgggtgccatgaatcacacctgcaatcccagcactttggaaatctgtggtgagaggatctcttaaagctatggccttgaaaccagcctgggtaacaaaaccaagcccttgtatctaaaaaacaaaagggcaggtagattccagataaataatgttgcaggaagggggtgataaaatacagaaatgtgatacacagcaaaccctcaactgctgtttcattggctcccagtgtcttaatctgtgtctgctaacactttaaggtacatctgaaataccccccaaacccagaaagcttttcaacagctaggttgtccaagaacttggaaaattcaccttctgatgtcctccaagacagattcc

SEQ ID NO: 3 - DNA, 40 bp, Homo sapiens
attactagagtgccgctttcagcccctctgtcgtcgccga

SEQ ID NO: 4 - DNA, 40 bp, Homo sapiens
tcggcgacgacagaggggctgaaagcggcactctagtaat

(19) United States
(12) Patent Application Publication
(10) Pub. No.: US 2021/0131968 A1
(43) Pub. Date: May 6, 2021

(54) IMAGING-DIRECTED NANOSCALE PHOTO-CROSSLINKING

(71) Applicant: University of Southern California, Los Angeles, CA (US)

(72) Inventors: Lin Chen, Los Angeles, CA (US); Yi Kou, Los Angeles, CA (US)

(21) Appl. No.: 16/963,167

(22) PCT Filed: Jan. 25, 2019
(86) PCT No.: PCT/US2019/015093

§ 371 Date: Jul. 17, 2020

Related U.S. Application Data
(60) Provisional application No. 62/622,044, filed on Jan. 25, 2018.

(51) Int. Cl. G01N 21/64; G01N 27/447; G01N 23/22

(52) U.S. Cl. CPC G01N 21/6458; G01N 23/22; G01N 27/44791; G01N 27/44721

(57) ABSTRACT

A method to induce photo-chemical reactions in a nanoscale space is provided. The method includes fixing cells and incubating the cells with a probe containing a tag for a click reaction. The probe is a psoralen probe that includes an alkyne tag. The method further includes illuminating the cells with UV light on a cell nucleus in a selected region, incubating the cells with a click reaction mix that includes rhodamine-azide, clicking the azide to the psoralen probe through its terminal alkyne, removing excess rhodamine, and viewing the cells with a fluorescence microscope.

### CROSS-REFERENCE TO RELATED APPLICATIONS

This application claims benefit of priority under 35 U.S.C. § 119(e) of U.S. Ser. No. 62/622,044, filed Jan. 25, 2018, the entire contents of which is incorporated herein by reference in its entirety.

### STATEMENT REGARDING FEDERALLY SPONSORED RESEARCH AND DEVELOPMENT

This invention was made with government support under Contract Nos. 5U54DK107981 and A111300901A1 awarded by the National Institute of Health. The government has certain rights in the invention.

### INCORPORATION OF SEQUENCE LISTING

The material in the accompanying sequence listing is hereby incorporated by reference into this application. The accompanying sequence listing text file, name USC1380_1WO_Sequence_Listing.txt, was created on Jan. 24, 2019, and is 3 kb. The file can be accessed using Microsoft Word on a computer that uses Windows OS.

### FIELD OF THE INVENTION

The present invention relates to methods and systems for determining structural information within cells.

### BACKGROUND OF THE INVENTION

Imaging analyses have long established that the 3D structure of the nucleus and its dynamic nature are closely related to cellular functions. However, it is not until recently that genome-wide analyses of the nuclear structure started to reach the molecular level. Studies suggest that direct physical models of the genome can be generated from extensive mapping of chromatin interactions and population-based modeling and that the resulting models can yield insights about genomic functions via statistical analyses. While these studies provide a glimpse of the great potential of understanding cellular functions from the molecular structures of the nucleus, it remains a major challenge to develop an accurate physical model of the nucleus in space and time and relate the model structures to cellular functions. Thus, there is a need to develop comprehensive and robust approaches to structural analyses of the nucleus.

It is well known that cells contain sub-cellular/sub-nuclear compartments and foci with distinct functions and molecular compositions (protein, DNA, RNA and other bio-molecules). and sub-nuclear. The small volume (usually around 100s nanometer scale) and dynamic nature of these compartments and foci make it challenging to probe the molecular content of these sub-cellular/sub-nuclear compartments and foci and their link to physiological functions and diseases. There is no technology available to determine the molecular content in a nanoscale sub-cellular and sub-nuclear space at a specific time point. Thus, there is a need to develop such technology.

### SUMMARY OF THE INVENTION

One aspect of the present invention is to provide a method to induce photo-chemical reactions in a nanoscale space. The method includes incubating the cells with a cell permeable probe containing a photo-crosslinking functional group and a tag for a click reaction, illuminating the cells with UV light on a cell nucleus in a selected region, and incubating the cells with a click reaction mix.

In one embodiment, the probe is a psoralen probe containing an alkyne tag.

In another embodiment, the reaction mix includes rhodamine-azide.

In another embodiment, the method further includes clicking the azide to a psoralen probe through its terminal alkyne.

In another embodiment, the method further includes removing excess rhodamine; and viewing the cells with a fluorescence microscope.

In one embodiment, the reaction mix includes biotin-azide.

In another embodiment, the method further includes tethering DNA from the UV illuminated region using a streptavidin bead.

In another embodiment, the method further includes pulling down and sequencing the DNA after clicking the azide to a psoralen probe.

Another aspect of the present invention is to provide a method to induce photo-chemical reactions in a nanoscale space. The method includes fixing cells; incubating the cells with a probe containing a tag for a click reaction, wherein the probe is a psoralen probe comprising an alkyne tag; illuminating the cells with UV light on a cell nucleus in a selected region; incubating the cells with a click reaction mix, wherein the click reaction mix includes rhodamine-azide; clicking the azide to the psoralen probe through its terminal alkyne; removing excess rhodamine; and viewing the cells with a fluorescence microscope.

Another aspect of the present invention is to provide a method to induce photo-chemical reactions in a nanoscale space. The method includes fixing cells; incubating the cells with a probe containing a tag for a click reaction, wherein the probe is a psoralen probe comprising an alkyne tag; illuminating the cells with UV light on a cell nucleus in a selected region; incubating the cells with a click reaction mix, wherein the click reaction mix includes biotin-azide; tethering DNA from the UV illuminated region using a streptavidin bead; clicking the azide to the psoralen probe through its terminal alkyne; and pulling down and sequencing the DNA.

Another aspect of the present invention is to provide a method for designing probes for probing DNA and RNA in a specific nano-space inside cells. The method includes selecting a small molecule that binds DNA and/or RNA; and introducing a photo-affinity label and an alkyne tag into the small molecule.

In one embodiment, the small molecule is selected from the group that includes psoralen, DAPI, polyamide and any small molecule that binds DNA and/or RNA non-specifically and/or specifically.

In another embodiment, the photo-affinity label includes azido, diazirine and benzophenone.

Another aspect of the present invention is to provide a method for designing probes for probing proteins in a specific nano-space inside cells. The method includes selecting a small molecule that binds proteins; and introducing a photo-affinity label and an alkyne tag into the small molecule.

In one embodiment, the photo-affinity label includes azido, diazirine and benzophenone.

Other aspects and advantages of the invention will be apparent from the following description and the appended claims.

### BRIEF DESCRIPTION OF THE DRAWINGS

FIG. 1. Schematic of illumination process.

FIG. 2. Steps of illumination process.

FIGS. 3A-3B. (A) Parts of INPX probe. (B) Examples of chemical structures of probe.

FIGS. 4A-4C. (A) Hela cell under bight field. Arrow: illuminated areas. (B) After UV illumination. (C) Cells in fluorescence microscope.

FIGS. 5A-5C. (A) and (B) Framed: areas under two photon illumination. (C) After clicked to rhodamine fluorophore and washed.

FIGS. 6A-6B. (A) Hela cell under bright field with UV laser (arrow). (B) After UV illumination.

FIG. 7. Schematic of selected DNA sequence pull-down and cut off using psoralen probe.

FIG. 8. Chromatogram alignments for sequence comparison. 7-6_S13 (upper chromatogram): active euchromatin from cut-off DNA; E111 Hela-S3 Cervi (lower chromatogram): negative DNA control.

FIG. 9. Schematic of the steps for illumination and capture of RNA molecules.

FIG. 10. Electrophoresis gels showing the PCR amplification products of the capture sequences. Left: 4 different capturing DNA for SNHG1 lncRNA; right: negative capturing control.

FIG. 11. Sequences of the captured RNA molecules. Reverse complement: SNHG1 lncRNA capturing DNA. SEQ ID NO. 1: Reverse complement sequence amino acid 1-420; SEQ ID NO. 2: Reverse complement sequence amino acid 421-1081; SEQ ID NO. 3: negative control sequence; SEQ ID NO. 4: Negative control reverse complement sequence.

### DETAILED DESCRIPTION OF THE INVENTION

Before the present methods and compositions are described, it is to be understood that this invention is not limited to particular methods, compositions, and experimental conditions described, as such methods, compositions, and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. The definitions set forth below are for understanding of the disclosure but shall in no way be considered to supplant the understanding of the terms held by those of ordinary skill in the art.

As used in this specification and the appended claims, the singular forms “a”, “an”, and “the” include plural references unless the context clearly dictates otherwise. Thus, for example, references to “the method” include one or more procedures/methods, and/or steps of the type described herein which will become apparent to those persons skilled in the art upon reading this disclosure and so forth.

The present invention uses a new technology that uses a laser to induce photo-chemical reactions in a nanoscale space guided by microscope imaging. The general idea is to apply a custom designed photo-chemical probes in a medium, and then use a laser of proper wavelength to focus on a selected volume, guided by microscope imaging, to induce photo-chemical reactions in a nanoscale volume. The photo-chemical reaction can be used to modify the properties of selected volume in the medium. This technology has applications in material design and engineering. This technology can also be used to identify cellular and genomic information in a nanoscale sub-cellular and sub-nuclear space at a specific time point.

As previously discussed, there is no technology available to determine the molecular content in a nanoscale sub-cellular and sub-nuclear space at a specific time point. The inventors have developed a general strategy of imaging-directed nanoscale photo-crosslinking to achieve this goal. First, small chemical probes were designed and synthesized that bind DNA and protein either non-specifically or specially, and are cell permeable and non-toxic (at least for the duration of the imaging-directed nanoscale photo-crosslinking (INPX) experiment). The probes can be activated by long wavelength of UV (330-370 nm) that specifically activate the photo-crosslinking of the probes but do not damage cellular proteins, DNA or RNA. The probes are also engineered to have affinity tags for subsequence enrichment of photo-crosslinking captured DNA, RNA and proteins. One design of the tag is introducing an alkyne moiety on the probe. After the photo-crosslinking reaction, proteins, DNA, RNA covalent crosslinked to the probe can be enriched by click-chemistry using azido-biotin that can react with alkyne. Other strategies of chemical conjugation may also be applied. Modern laser technology is used to focus on a small volume (less than 200 nm×200 nm×200 nm) that can be selected by proper molecular markers (e.g., GFP-labeled proteins, genomic region identified by FISH probes etc.). Two-photon laser technology can be used to illuminate selected nano-volumes. The laser intensity is adjusted so that the photo-crosslinking reaction can be completed with high efficiency without damaging the cells. The crosslinked protein can be isolated using the affinity tag and identified with known methods such as mass spectrometry or antibody-based methods. The crosslinked DNA and RNA can be isolated using the affinity tag and identified with known sequencing methods (e.g., single molecule protein detection techniques and single molecule DNA/RNA sequencing.)

INPX has the following unique features to bring a revolutionary technology to the fields of nanomaterial sciences and biological sciences: i) by combining high resolution microscope imaging, laser technologies, custom-designed photo-chemical molecules, photo-chemical reaction can be induced in a selected nano-volume instantly; (ii) the nanoscale spatial resolution of laser focus and the sub-second (down to femtosecond) temporal resolution of laser pulse can allow unprecedented spatial/temporal control of chemical reactions for material design and for information capture. The following examples describe the application of INPX in capturing the molecular information in a specific nano-volume inside cell, including cytoplasmic space, nuclear space, membrane boundaries or any sub-cellular/sub-nuclear compartments or foci of interest.

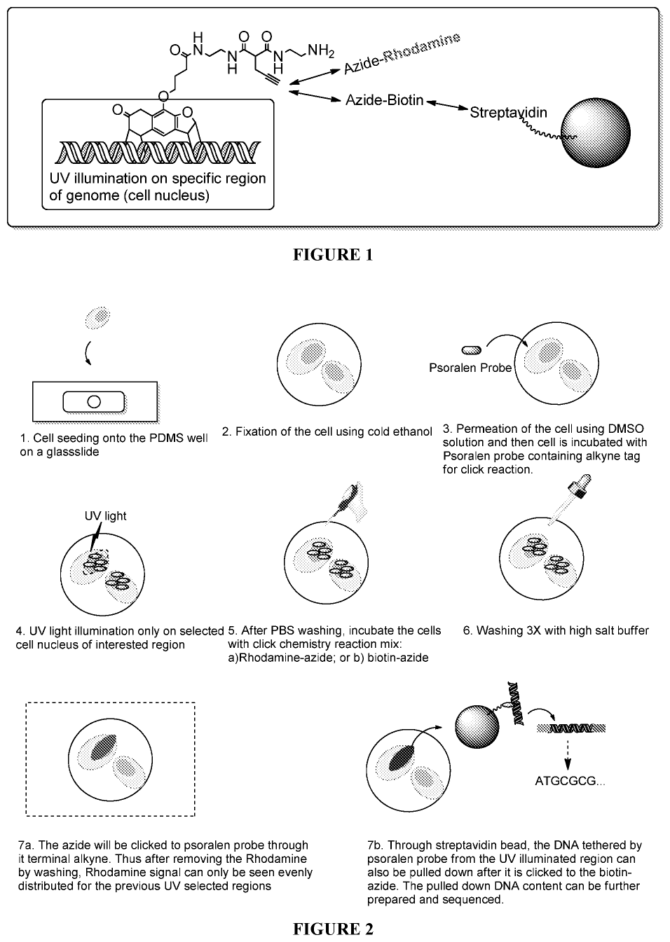

Assisted by imaging, INPX can extract various DNA/RNA/protein information at the subcellular or subnucleus site at will. For example, to extract the genomic information around the observation site, a psoralen probe will be firstly incubated with the cell nucleus. Then UV illumination will only be applied to the observation site. By photocrosslinking, only the DNA in the UV illuminated region will be linked to psoralen probe. After removing the free probe by washing, the probe can then be linked to azide-biotin through click chemistry and the DNA it captures can thus be pulled down onto streptavidin beads for further analysis (e.g., sequencing). To confirm the applicability for certain cell type beforehand, the probe can also be clicked to azide-Rhodamine to be observed of its UV illumination pattern under microscope. The general process is illustrated in FIG. 2.

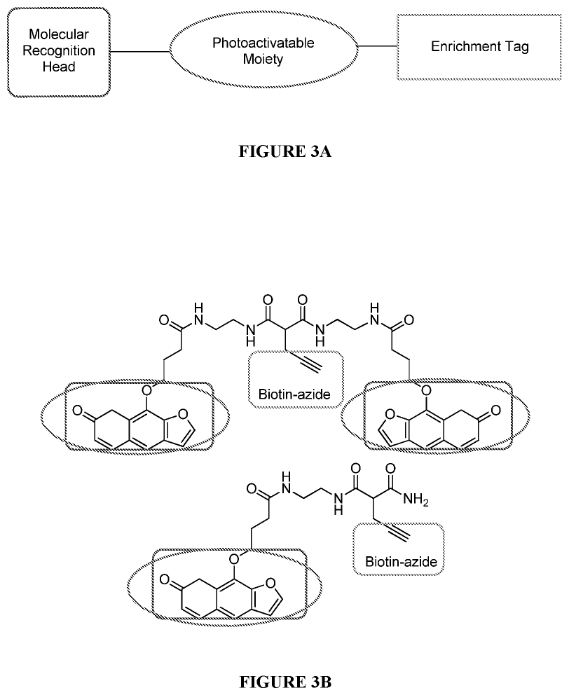

The general probe design is disclosed in FIGS. 3A and 3B. As disclosed in FIG. 3A, the probe of INPX consists of 3 function parts: the molecular recognition head which serves to recognize the target biomolecules inside the cells; a photoactivatable moiety, through which, the molecular recognition head can be photo actively tethered to the target biomolecules; and also, an enrichment tag, which serves the function of selecting out the probe together with the tethered biomolecules from the cell, enriching the sample for further analysis such as sequencing or mass spectrometry analysis.

In the examples of application section, the inventors have successfully demonstrated the applicability of both the bis-probe and half probe, as shown in FIG. 3B. As shown below, for the molecular recognition head it can be designed using psoralen, DAPI(2-(4-amidinophenyl)-1H-indole-6-carboxamidine) to capture DNA molecule. Additionally, polyamides or other DNA analogs can also be employed. As shown in FIG. 3B, for psoralen, obviously, it is both a molecular head and photoactivatable moiety, as it can recognize and capture DNA molecule under UV illumination. The inventors have also designed the maleimide molecular recognition head for capturing protein, because its reaction with sulfurhydryl group on any protein is well known. As shown below, for other INPX probes, the inventors have employed various bioorthogonal photoactivatable moiety and reactions. For the enrichment tag, as can be seen from the bis and half psoralen probes, it is designed in two parts: the two will be connected by click chemistry. And since the azide part contains the biotin, so the probes together with their captured biomolecules from the cell will be pulled down and thus enriched for further analysis (FIG. 2, step 7b). All of this chemistry has been established.

Psoralen Based DNA/RNA Capturing Probes

### i) Psoralen Based DNA/RNA Direct Capturing Probes:

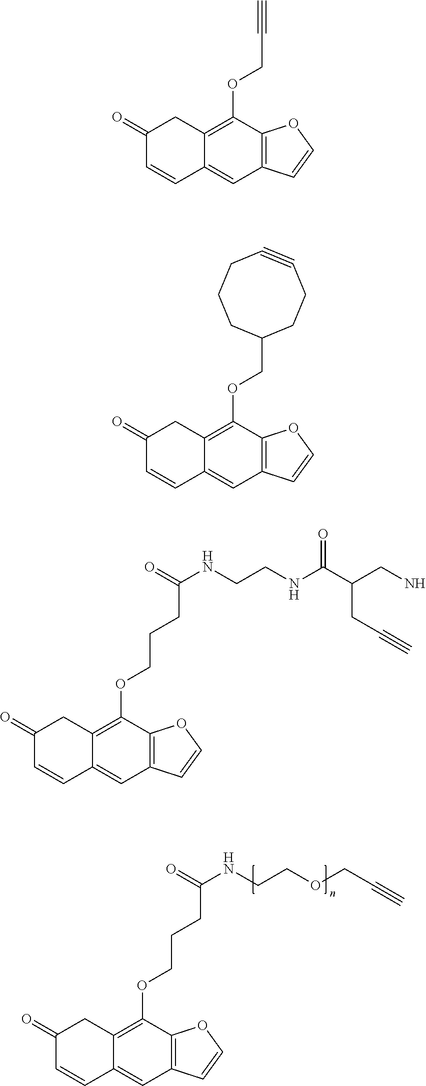

These probes can be used direct to tether DNA/RNA at wanted cell nucleus region using UV illumination, and the captured nucleic acids can be pulled down to streptavidin beads by further reacting with azide-biotin linker, since the alkyne tag on these probes can react with azide and biotin from the linker will be captured by streptavidin on the beads.

### ii) Psoralen Based DNA/RNA Indirect Capturing Probes:

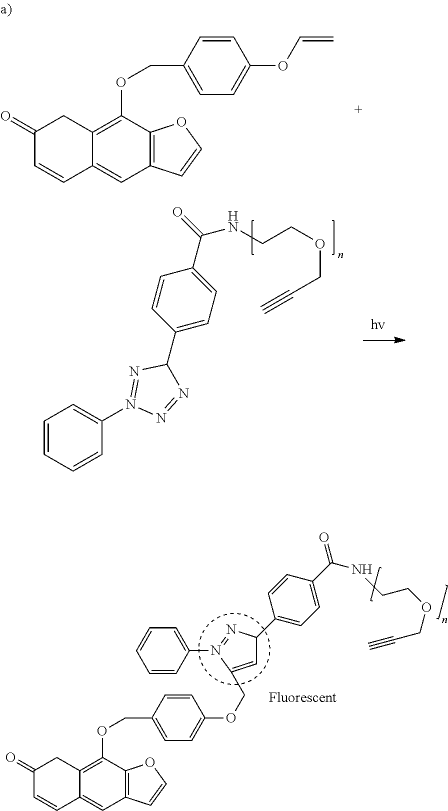

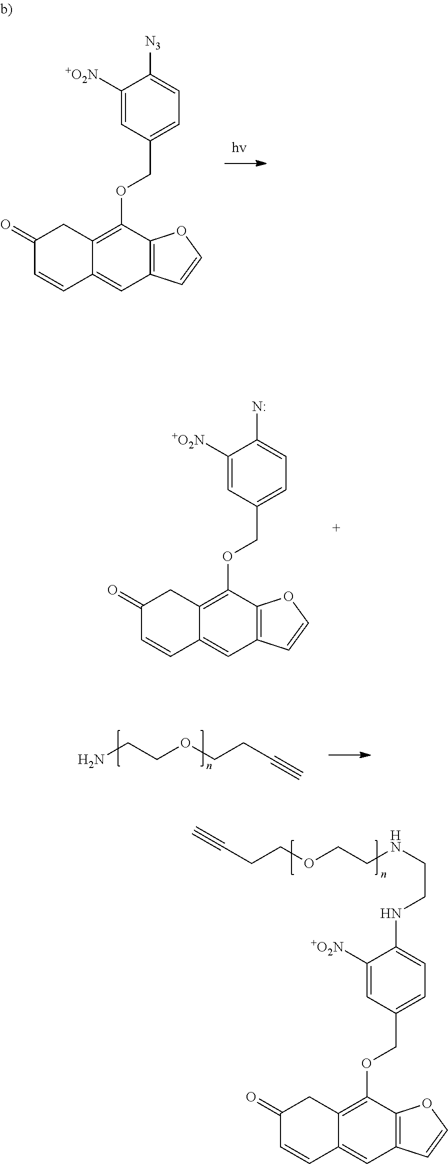

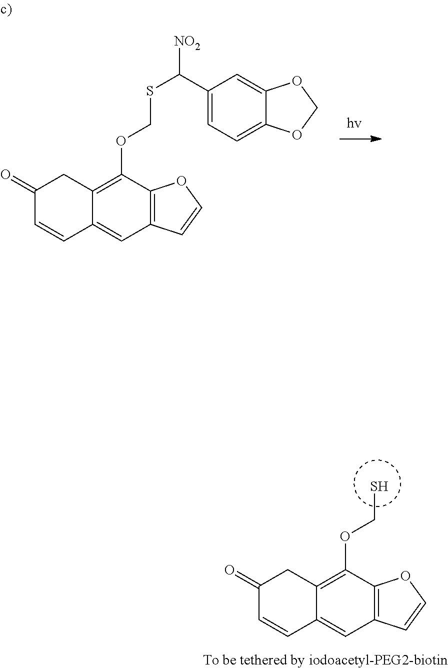 [SMILES: O=C1C=Cc2cc3ccoc3c(OCSC(c3ccc4c(c3)OCO4)[N+](=O)[O-])c2C1 ; O=C1C=Cc2cc3ccoc3c(OCS)c2C1]

As disclosed here, psoralen probes can also be designed to bear the alkyne/sulfurhydryl pull down tag through another photoreaction. In this design, psoralen probe can firstly bind to DNA/RNA under full nucleus/selective UV illumination, after washing the free probe, a second probe bearing the alkyne/sulfurhydryl tag would be diffused around. And under another illumination (around 300 nm), this second probe would be covalently linked to psoralen probe through photoreaction and confers the DNA/RNA bound psoralen probe the potential ability to be pulled down by streptavidin (through alkyne groups clicked to biotin azide as in A. and B., or sulfurhydryl group reacted with iodoacetyl-biotin as in C.) The two step photoreactions gives following advantages i) Double selection improves selection precision, decreases noise. ii) The other area bound by psoralen but not by the second probe can be pulled down later as background control. iii) After the second photoreaction, the final probe in A. will be fluorescent in situ, providing additional confirmation for pull down success.

DAPI Based DNA/RNA Capturing Probes:

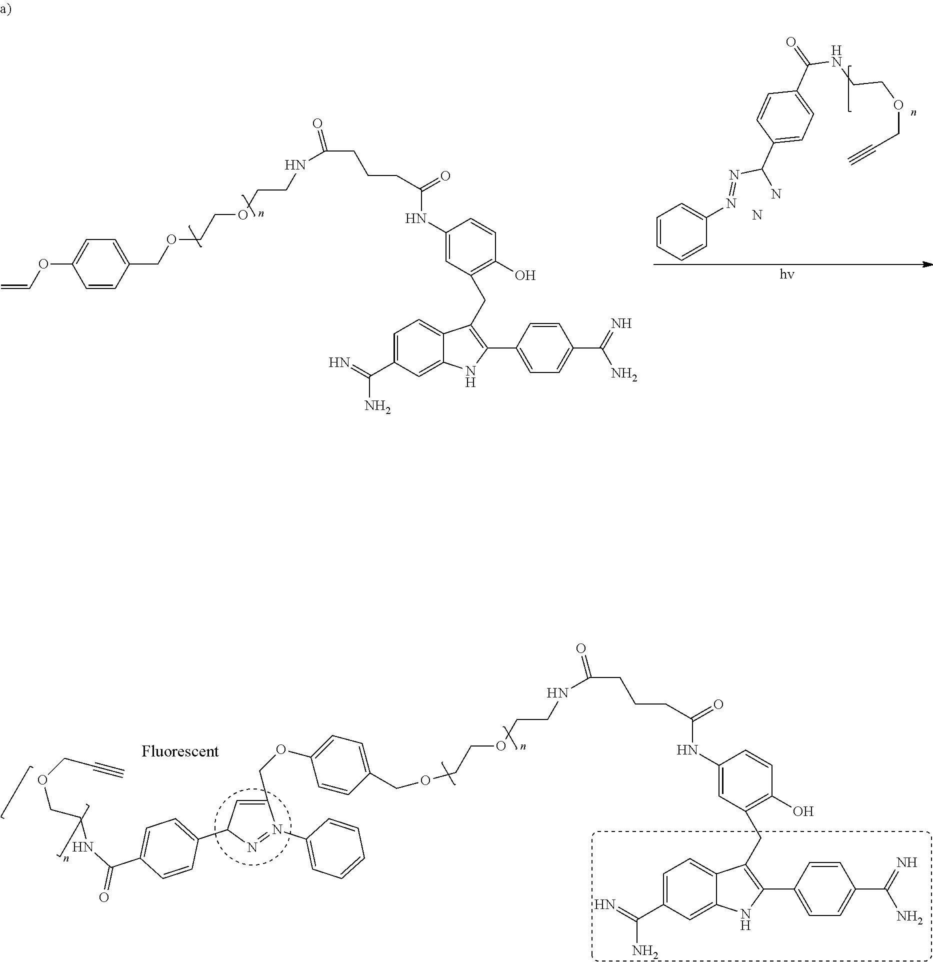

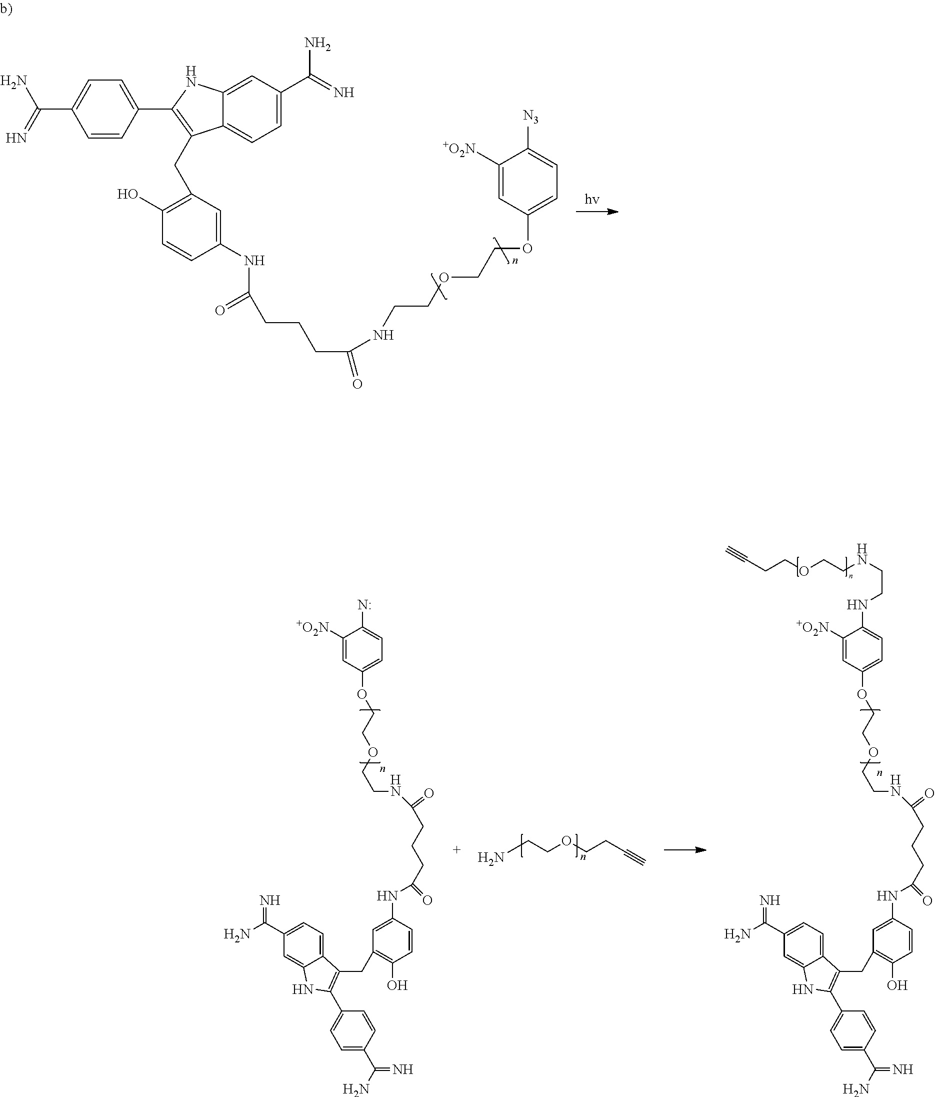

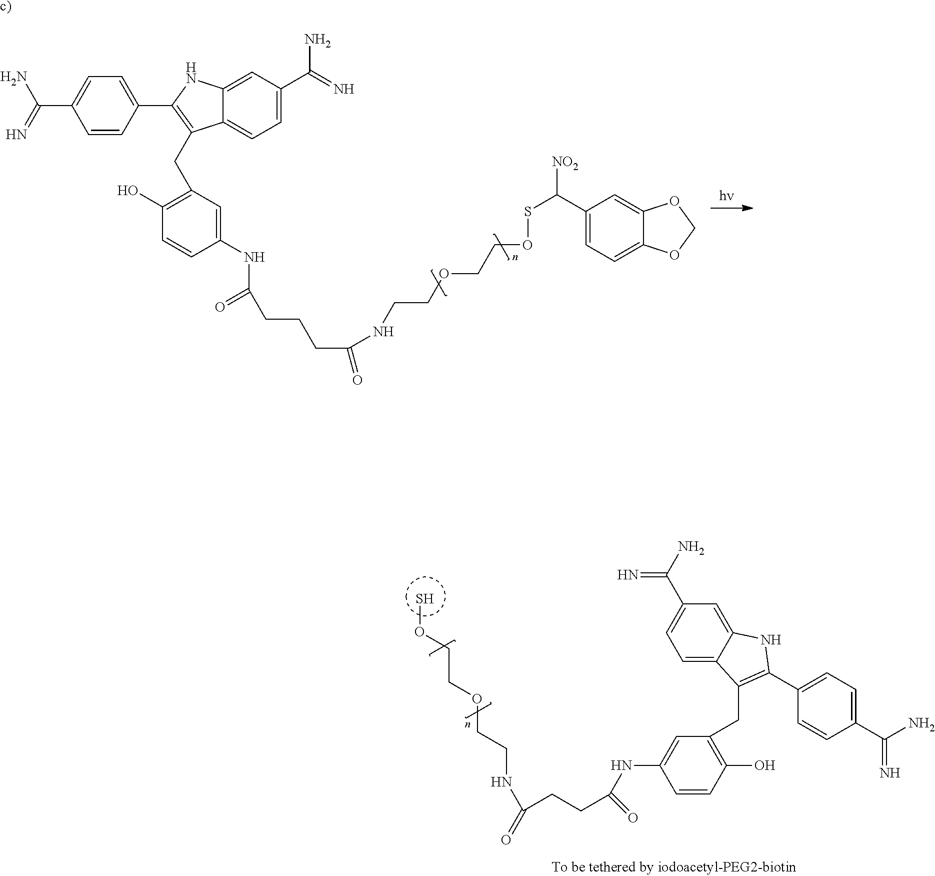

As disclosed here, DAPI is a well-known DNA minor groove binder. It has following advantages: i) It is solvable and diffuses evenly to nucleus DNA/RNA. ii) It has good fluorescent property, usually indicates much more clear structures in the cell nucleus for selection. iii) It binds to DNA tightly enough yet produces little effect for further DNA sequencing library preparation. As can be seen from the above, here, in A., B., and C., the similar second probe photo reaction is applied. The process is: cell nucleus incubated with these designed DAPI probes, then it will be incubated with these second probes. For specific wanted region in the nucleus, illumination will take place, and thus the photoreaction of connecting the second probe to the DAPI probe. Then alkyne groups or sulfurhydryl groups will be equipped and clicked to biotin azide or iodoacetyl biotin, and can thus be pulled down by the streptavidin beads through biotin-streptavidin linkage.

Maleimide Protein Capturing Probes

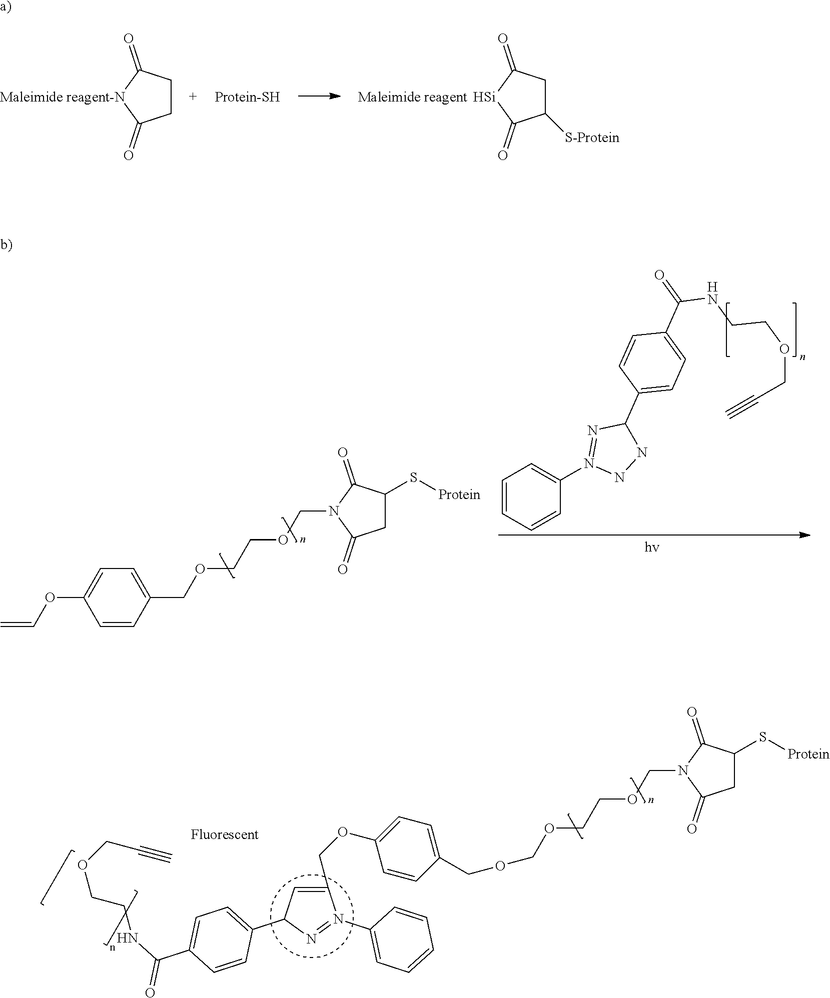

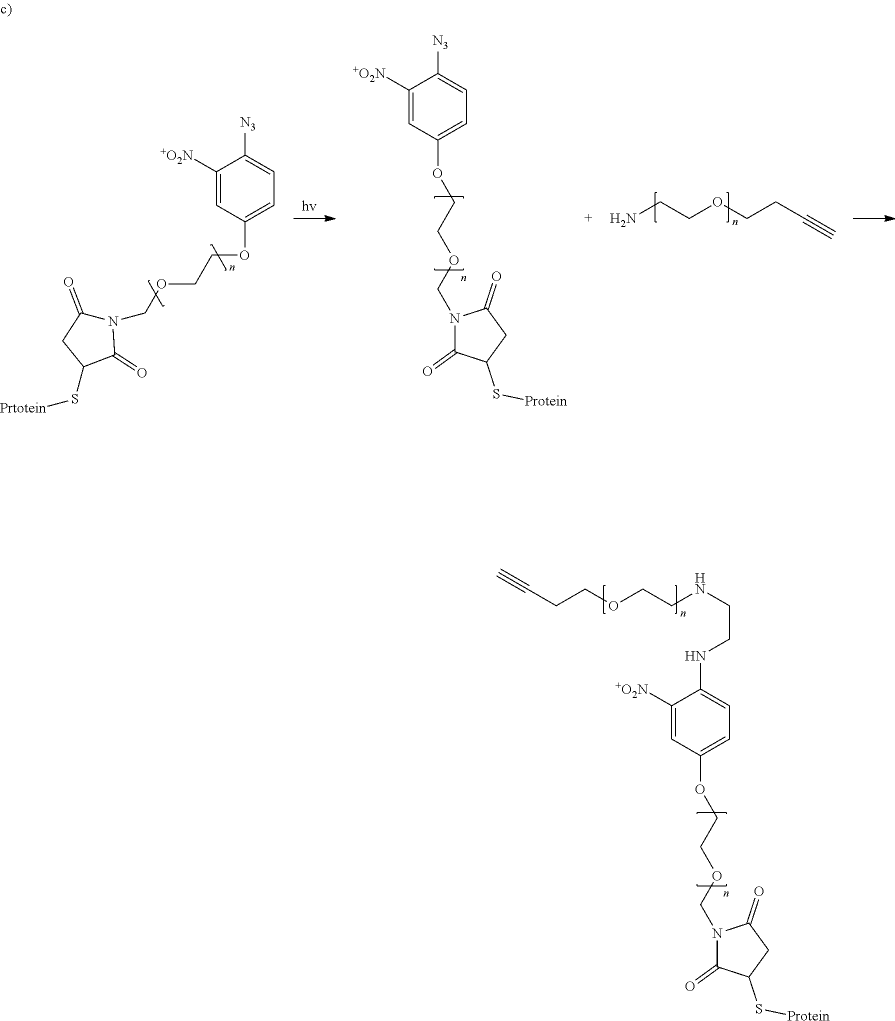

As disclosed in A, the maleimide group is known to react specifically with sulfhydryl groups on protein, the result is formation of a stable thioether linkage that is not reversible. Therefore, similar imaging assisted photoreaction probe can be designed for protein. This would work for the proteins in the whole cell, not only the cell nucleus. After the cell being incubated with probes shown in B. and C. (without protein S thioether link), a second probe will be added. And only for a specific wanted region on a cell, there will be enough illumination, and through azide biotin click chemistry, the protein from this region will be captured by the probe and pulled down by streptavidin beads. Proteins can then be submitted to various western, immunoprecipitation, or mass-spectrometry assays or to be further purified for their own usages.

### EXAMPLES

### Example 1

### Celluar Illumination

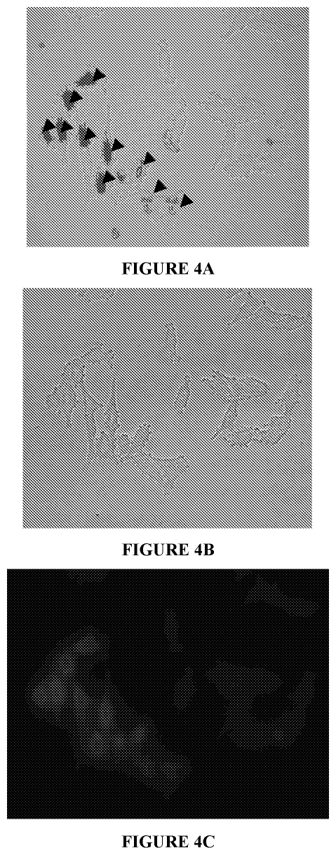

Using a UV laser microscope the design described in FIG. 2, steps 1 to 7a has successfully been applied to human cancer cell line Hela cell. The photo activation was performed by UV laser microscope: Solid-state, diode-pumped Q-switched (345 nm) with adjustable laser current and pulse frequency. FIG. 4A shows the Hela cells under bright field with a UV laser traced, as pointed out by the arrows. The highlighted areas (arrows) of the nuclei were illuminated with UV.

As shown in FIG. 4C, after UV illumination only highlighted areas (arrows) of the cells were later observed using fluorescence microscopy. The cells were then connected to the Rhodamine azide as in FIG. 2 step 5 by click chemistry. Then after thorough washing (step 6), the cells were observed under fluorescent microscope to check whether Rhodamine stayed at the place where we UV illuminated inside the cell nucleus. The results indicated that this design was successful: through the probe, only the nuclei that were UV illuminated were attached to a fluorophore.

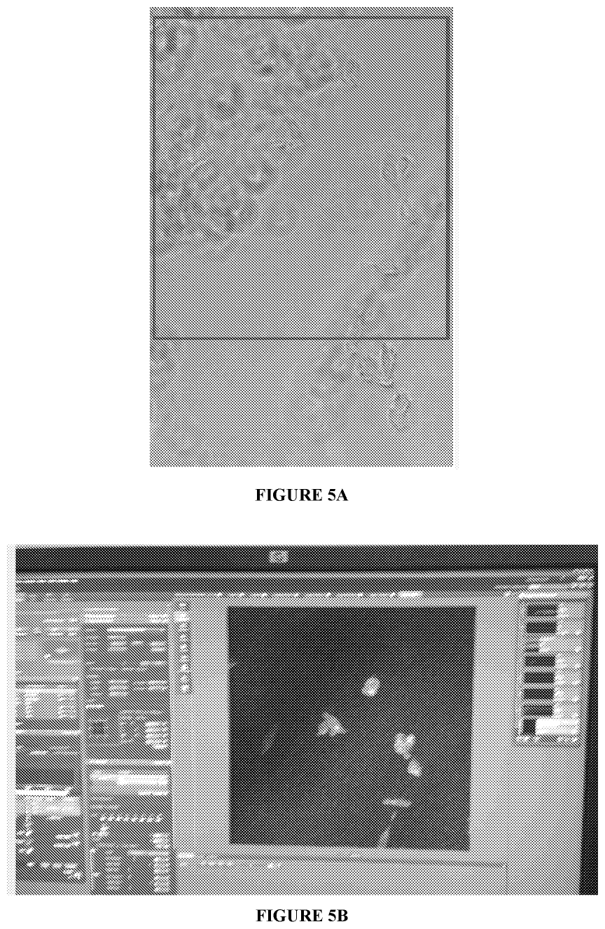

Further, it was also successfully proven that two photon microscope at 740 nm could generate around a 350 nm wavelength UV that could be used to connect the probe to the cell and later clicked with a fluorophore to show the fluorescence. FIGS. 5A and B illustrate that the chosen field within the framed area was under two photon illumination. The cells were then clicked to Rhodamine fluorophore and washed. The cells were then observed under fluorescent microscope as shown in FIG. 5C. This experiment shows that a two photon light source could be used to activate and tether the probe to the cell genome. Since the two photon light source can focus into a 200 nm*200 nm*200 nm cube in the 3D dimension, this INPX design can be applied to tether and eventually pull down the target biomolecules with a super resolution, which has not been achieved by any techniques before.

Using the same UV laser microscope discussed above, a half probe was applied to Hela cells (the previous two results were performed by applying the design described in FIG. 2 step 1 to 7a to human cancer cell line Hela cell and using the bis-probe).

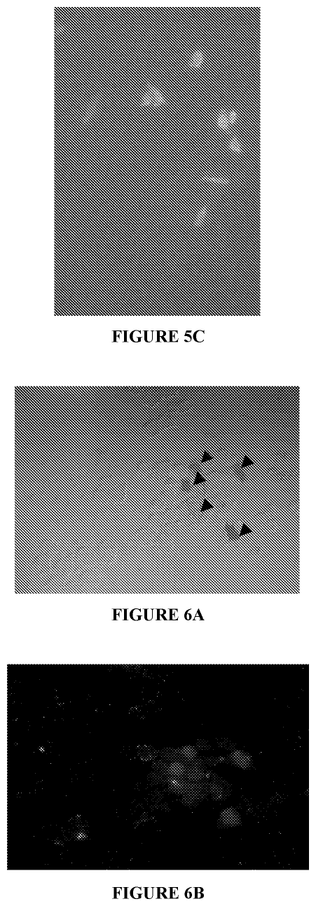

As shown in FIG. 6A, the Hela cell observed under bright field were illuminated with UV laser following the highlighted areas (arrows). Special patterns have been drawn to discern artificially made UV illumination pattern later under fluorescent detection. The cells at the time had been incubated with the probe. Then the cells were clicked with Rhodamine fluorophore and washed thoroughly. As illustrated in FIG. 6B, after UV illumination, only the cells with the UV illumination showed the exact pattern under expected signal channel. The bright dots were determined to be the contamination. The success of this assay testified the applicability of the design of the psoralen based probes.

### Example 2

### Selective Pull-Down and Cut-Off of DNA Sequence Using Psoralen Probe

Modified photo-activable molecules that bind to target biomolecules under the illumination of selected region were used for target biomolecule capture.

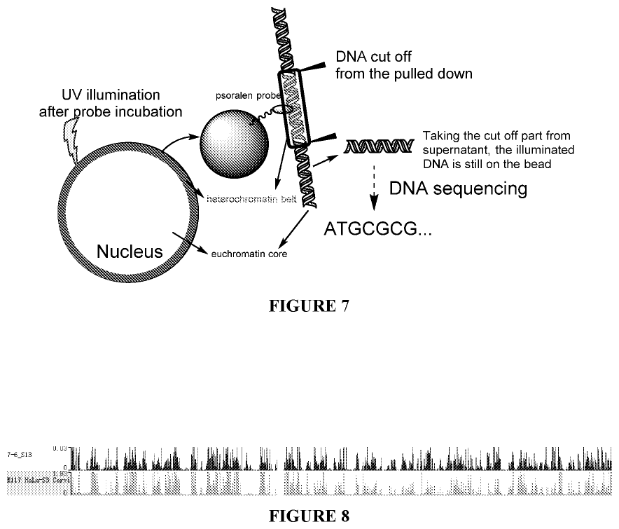

As illustrated in FIG. 7, the psoralen probes were modified to include a chemical tags for pulling-down, so that the probe and its captured bio-target can be enriched through the pull-down process. The subnuclear INPX was applied to extract the DNA from the targeted region. In addition, to amplifying the DNA directly from the illumination area, a reverse selection was also applied to confirm the selectivity from INPX. As described in FIG. 7, the whole cell nucleus was incubated with the psoralen probe. Then UV illumination was only applied to the heterochromatin region of the nucleus, which is the belt region near the edge. Beads were used to directly pull down the psoralen probe that was bound to DNA. Since the DNA had not yet gone through the restriction digestion, the whole chromosomes were pulled down together onto the bead, which included both the heterochromatin and euchromatin regions. Since the UV activation was done only to the heterochromatin belt, the psoralen probe was only bound directly to the DNA content in this region (shown in box). The following digestion of the DNA by restriction enzyme allowed the cut-off of DNA from other region: the euchromatin core was released into the supernatant, whereas the heterochromatin belt remained on the beads.

To further confirm the result, the cut-off DNA, was sequenced to verify its euchromatin core origin. As shown in the sequence correlation illustrated in FIG. 8, where the upper track (7-6_S13) is from the sequencing result of cut-off DNA, and lower track (E117 Hela-S3 Cervi) is the H3K4Me3 track from the same cell line, it correlated well (>0.8) with the DNA sequence which bore an active euchromatin marker H3K4Me3, which also confirmed the success of the selectivity of the INPX technology.

### Example 3

### Selective Pull-Down and Cut-Off of RNA Sequence Using Psoralen Probe

Since the psoralen probe binds to DNA, RNA and protein, the INPX technology was also used to assess its ability to capture RNA and proteins by adjusting the purification choice, so that the psoralen pull-down enriched either of these categories. RNA capture was implemented using the INPX, in order to determine the type of DNA that is around the RNA molecule in an area of interest.

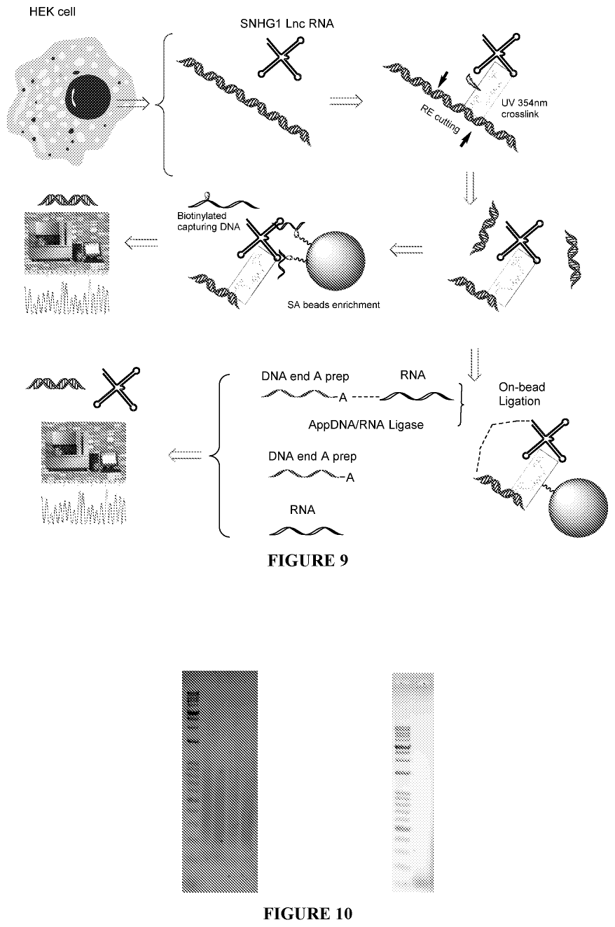

As illustrated in FIG. 9, after incubation of the cells with the psoralen probe, UV illumination was applied to certain regions of interest inside the nucleus. In this example, the target RNA was SNHG1 lncRNA, so a sequence-complementary capturing DNA was designed, which could capture the lncRNA by sequence matching and also be pulled down onto the streptavidin beads since it is biotinylated. After UV activation of the target area, and since bis-psoralen heads (two psoralen heads in one probe) were used, the psoralen probe was able to crosslink the nearby DNA and SNHG1 lncRNA. Specific SNHG1 capturing DNA and beads were then used to pull down all these (SNHG1 lncRNA and its nearby DNA target). Because the experiment aimed at capturing the nearby DNA, the SNHG1 lncRNA was digested by RNase, which released its nearby target DNA off from the beads to the supernatant.

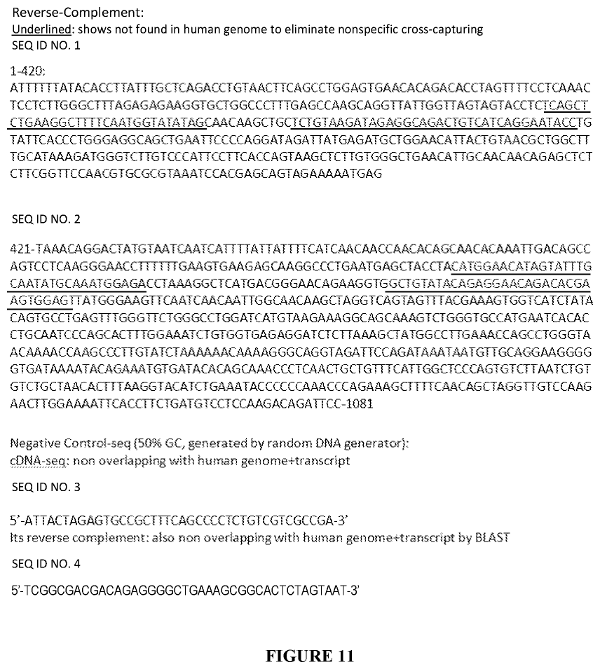

Four different capturing DNA for SNHG1 lncRNA were designed, and as illustrated in FIG. 10 (left) all of them successfully allowed the capture of DNA. More importantly, to double confirm INPX's selectivity, a negative control capturing DNA, whose sequence was not overlapped by any part of the human genome was also designed. As shown in FIG. 10 (right), even after PCR amplification, no DNA was captured, which confirmed the specificity of the assay. Capturing sequences were sequenced and aligned, and shown in FIG. 11. SEQ ID NO. 1 and SEQ ID NO. 2 corresponded to the reverse complement sequence of SNHG1 lncRNA capturing DNA, from amino acid 1-420 and 421-1081 respectively; and with underlined sequences referring to sequence not found in human genome, included to eliminate non-specific cross capturing. SEQ ID NO. 3 and SEQ ID NO. 4 respectively referred to the sequence and the reverse complement sequence of the negative capturing control.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

All references cited herein, including those below and including but not limited to all patents, patent applications, and non-patent literature referenced below or in other portions of the specification, are hereby incorporated by reference herein in their entirety.
1) PCT/US17/65418, filed Dec. 8, 2017.

What is claimed is:

1. A method to induce photo-chemical reactions in a nanoscale space comprising:
using live or fixed cells;
incubating the cells with a probe containing photo-crosslinking functional group and a tag for a click reaction;
illuminating the cells with UV light on a cell nucleus in a selected region;
and incubating the cells with a click reaction mix.

2. The method of claim 1, wherein the probe is a psoralen probe containing an alkyne tag.

3. The method of claim 1, wherein the reaction mix comprises rhodamine-azide.

4. The method of claim 1, wherein the reaction mix comprises biotin-azide.

5. The method of claim 3, further comprising clicking the azide to a psoralen probe through its terminal alkyne.

6. The method of claim 5, further comprising removing excess rhodamine; and viewing the cells with a fluorescence microscope.

7. The method of claim 4, further comprising tethering DNA from the UV illuminated region using a streptavidin bead.

8. The method of claim 7, further comprising pulling down and sequencing the DNA after clicking the azide to a psoralen probe.

9. A method to induce photo-chemical reactions in a nanoscale space comprising:
fixing cells;
incubating the cells with a probe containing a tag for a click reaction, wherein the probe is a psoralen probe comprising an alkyne tag;
illuminating the cells with UV light on a cell nucleus in a selected region;
incubating the cells with a click reaction mix, wherein the click reaction mix comprises rhodamine-azide;
clicking the azide to the psoralen probe through its terminal alkyne;
removing excess rhodamine; and
viewing the cells with a fluorescence microscope.

10. A method to induce photo-chemical reactions in a nanoscale space comprising:
fixing cells;
incubating the cells with a probe containing a tag for a click reaction, wherein the probe is a psoralen probe comprising an alkyne tag;
illuminating the cells with UV light on a cell nucleus in a selected region;
incubating the cells with a click reaction mix, wherein the click reaction mix comprises biotin-azide;
tethering DNA from the UV illuminated region using a streptavidin bead;
clicking the azide to the psoralen probe through its terminal alkyne; and
pulling down and sequencing the DNA.

11. A method for designing probes for probing DNA and RNA in a specific nano-space inside cells comprising:
selecting a small molecule that binds DNA and/or RNA; and
introducing a photo-affinity label and an alkyne tag into the small molecule.

12. The method of claim 11, wherein the small molecule is selected from the group consisting of psoralen, DAPI, polyamide and any small molecule that binds DNA and/or RNA non-specifically and/or specifically.

13. The method of claim 11, wherein the photo-affinity label is selected from the group consisting of azido, diazirine and benzophenone.

14. A method for designing probes for probing proteins in a specific nano-space inside cells comprising:
selecting a small molecule that binds proteins; and
introducing a photo-affinity label and an alkyne tag into the small molecule.

15. The method of claim 14, wherein the photo-affinity label is selected from the group consisting of azido, diazirine and benzophenone.
